(19) United States
(12) Patent Application Publication
(10) Pub. No.: US 2018/0359994 A1
(43) Pub. Date: Dec. 20, 2018

(54) K-9 COOLIE

(71) Applicant: John McQuien, El Paso, TX (US)

(72) Inventor: John McQuien, El Paso, TX (US)

(21) Appl. No.: 15/622,178

(22) Filed: Jun. 14, 2017

(51) Int. Cl. A01K 13/00; A61F 7/02

(52) U.S. Cl. CPC A01K 13/006; A61F 7/02; A61F 2007/0234; A61F 2007/023; A61F 2007/0219

(57) ABSTRACT

The K-9 Coolie is the only product of its kind that features an insulated mesh wrap that can be adjusted to fit any size of canine and keep pets perfectly cool for any extended period of time. The K-9 Coolie is manufactured with a lightweight, insulated mesh material for added durability and holds up to five frozen gel packs at a time for maximum comfort and protection for even the largest of canines.

### BACKGROUND OF THE INVENTION

### Field of the Invention

The present invention is an article of clothing for pets. More specifically, the present invention is an insulated mesh wrap for dogs.

### Description of the Related Art

There are currently no feasible products on the market that keeps pets perfectly cool in hot and humid temperatures. The heat and humidity outside can often be too much to bear for dogs during extended periods of time such as when owners are hosting a barbecue, family gathering out on the patio, receptions, camping trip, or just going to the park to play for a few hours. Moreover, similar products on the market are available with an evaporative cooler that is submerged in water, but fail to keep pets comfortably cool for extended periods of time.

### SUMMARY OF THE INVENTION

The present invention is an article of clothing for pets. More specifically, the present invention is a wrap for dogs.

The wrap includes pockets, gel packs, and VELCRO® straps.

It is an object of the present invention to keep canines of all sizes comfortably cool in virtually any hot and humid climate for a much longer period of time than its competitors.

It is an object of the present invention to provide a wrap that can be adjusted to fit any size of canine and keep the pet perfectly cool for any extended period of time.

It is an object of the present invention to provide a wrap that is made of a lightweight, insulated mesh material for added durability.

It is an object of the present invention to provide up to 5 frozen, non-toxic, removable gel packs at a time for maximum comfort and protection for even the largest of canines.

It is an object of the present invention to provide a secondary version in which the mesh wrap can be reversed if you want to keep your canine warm instead of cool.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates a front view of an insulated mesh wrap, in accordance with one embodiment of the present invention.

FIG. 2 illustrates a front view of a gel pack, in accordance with one embodiment of the present invention.

FIG. 3 illustrates a front view of a larger size gel pack than that in FIG. 2, in accordance with one embodiment of the present invention.

FIG. 4 illustrates a side view of a dog wearing the wrap, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention, however the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

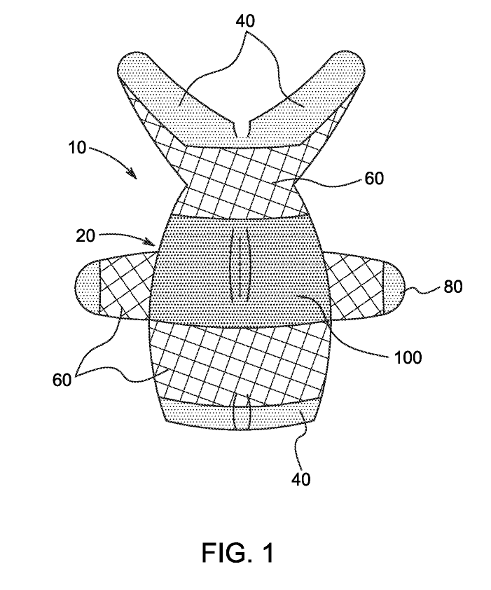

Referring now to FIG. 1, illustrated is a front view of the insulated mesh wrap 10, in accordance with one embodiment of the present invention.

In FIG.1, illustrated is a front view of the insulated mesh wrap 10, including a body 20, smaller gel packs 40 that are inserted in pockets of the wrap body, light-weight, mesh material 60, VELCRO® strap 80 for adjusting the wrap on the body of the dog, and larger gel packs 100.

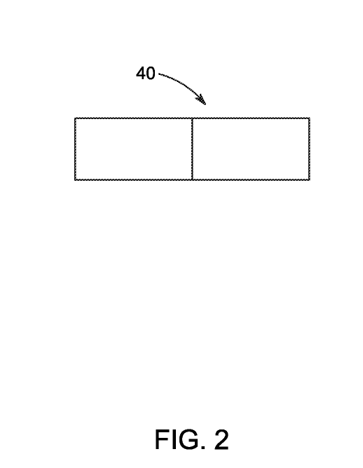

Referring now to FIG. 2, illustrated is a front view of the smaller gel packs 40, in accordance with one embodiment of the present invention.

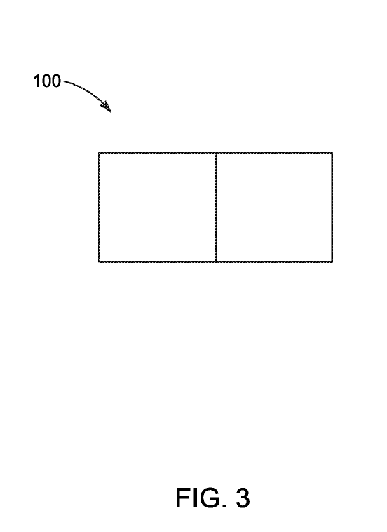

Referring now to FIG. 3, illustrated is a front view the larger gel packs, 100, in accordance with one embodiment of the present invention.

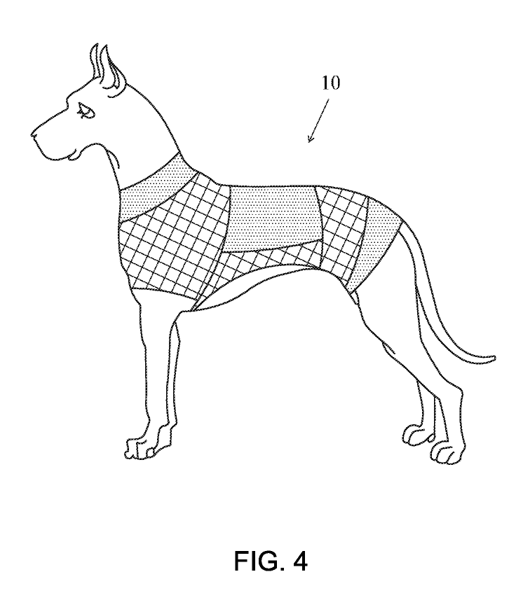

Referring now to FIG. 4, illustrated is a side view of a dog wearing the insulated mesh wrap with the gel packs, in accordance with one embodiment of the present invention.

While the present invention has been related in terms of the foregoing embodiments those skilled in the art will recognize that the invention is not limited to the embodiments described. The present invention may be practiced with modification and alteration within the spirit and scope of the appended claims. Thus, the description is to be regarded as illustrative instead of restrictive on the present invention.

What is claimed is:

1. A wrap for dogs, said wrap comprising:
a body;
a pocket, said pocket located on said body; and
a gel pack, said gel pack located in said pocket; and a
VELCRO® strap for adjusting the wrap on a body of a dog, said VELCRO® strap located on said wrap body.

2. The wrap for dogs according to claim 1, wherein said body is made of a lightweight mesh material.

3. The wrap for dogs according to claim 1, wherein said body is insulated.

4. The wrap for dogs according to claim 1, wherein said pocket comprises a plurality of pockets.

5. The wrap for dogs according to claim 4, wherein said plurality of pockets is up to five pockets.

6. The wrap for dogs according to claim 1, wherein said gel pack comprises a plurality of gel packs.

7. The wrap for dogs according to claim 6, wherein said plurality of gel packs is up to five gel packs.

8. The wrap for dogs according to claim 7, wherein said plurality of gel packs is non-toxic.

9. The wrap for dogs according to claim 1, wherein the mesh wrap can be reversed if you want to keep your canine warm instead of cool.
